(19) United States
(12) Patent Application Publication
(10) Pub. No.: US 2004/0166068 A1
(43) Pub. Date: Aug. 26, 2004

(54) Antiplaque denture adhesive compositions

(75) Inventors: Jayanth Rajaiah, Loveland, OH (US); Mark Ieuan Edwards, Langdale Close (GB); Ann Maria Kneipp, Cincinnati, OH (US); Jeanine Leslie Winston, Maineville, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/370,909

(22) Filed: Feb. 20, 2003

(51) Int. Cl. A61K 7/16; A61K 6/00

(52) U.S. Cl. 424/49; 523/120

(57) ABSTRACT

The present invention relates to a denture adhesive composition comprising a safe and effective adhesive amount of denture adhesive component; a safe and effective amount of an antiplaque/antistain agent selected from the group consisting of dimethicone copolyol, silicone surfactant, aminoalkylsilicone, organosiloxane resin, and mixtures thereof; and a non-aqueous denture adhesive carrier. The present invention further relates to a method of increasing the antiplaque, antistain, and/or antideposition efficacy or effect in the oral cavity of a denture wearer, in need thereof, by applying the above composition to the oral cavity.

### TECHNICAL FIELD

The present invention relates to a non-aqueous denture adhesive composition comprising a safe and effective adhesive amount of denture adhesive component and a safe and effective amount of dimethicone copolyol, silicone surfactant, aminoalkylsilicone, and/or organosiloxane resin, as an antiplaque/antistain agent.

### BACKGROUND OF THE INVENTION

Ordinary removable dentures, dental plates and the like, comprise teeth mounted in a suitable plate or base. Denture stabilizers are used to fill the interstices between the dentures and the gums or tissues. Prior to placement of the denture in the oral cavity, a denture stabilizer can be applied to the denture-plate surface. Considerable effort has been made in the past to develop improved denture adhesive compositions. The denture stabilizer is generally formulated not only for its adherent properties, but also to provide a cushion or gasket between the denture and the gums or tissues, thereby positioning the denture securely in the oral cavity. Both synthetic and natural polymers and gums have been used singly, in combination, and in combination with various adhesives and other materials in an attempt to lessen certain deficiencies. These deficiencies include inadequate holding power, oozing of the adhesive from under the dental plate during insertion and throughout the wearing period, and messiness and difficulty of removing the residual adhesive from the mouth and dentures.

Other problems are also associated with wearing dentures. Plaque, mucilaginous deposits, and bacterial deposits, resulting from food trapped in the denture, etc., can accumulate on dentures while being worn. Dentures with plaque and bacterial deposits can, within a short time, result in a detrimental effect on the mucous membranes and can result in malodor-formation. Plaque and bacterial deposits can also lead to so-called bacterial corrosion of the plastic material with possible color-change.

Certain silicones are known for use in denture cleansing compositions and oral care products such as dentifrice and mouthrinse for their ability to coat teeth, prevent caries and prevent staining. For instance, GB-A-689,679 discloses a mouthwash containing an organopolysiloxane for preventing adhesion of, or for removing tars, stains, tartar and food particles from the teeth. U.S. Pat. No. 2,806,814 discloses dental preparations including, in combination, a higher aliphatic acyl amide of an amino carboxylic acid compound and a silicone compound. Dimethyl polysiloxanes are said to be particularly effective. U.S. Pat. No. 3,624,120 discloses quaternary ammonium salts of cyclic siloxane polymers for use as cationic surfactants, bactericides, and as anticariogenic agents. Other references include U.S. Pat. No. 5,759,523, Hughes et al, issued Jun. 2, 1998; U.S. Pat. No. 5,827,505, Hughes et al., issued Oct. 27, 1998; U.S. Pat. No. 6,004,538, Hughes et al., issued Dec. 21, 1999; U.S. Pat. No. 5,154,915, Weber et al., issued Oct. 13, 1992; U.S. Pat. No. 5,427,770, Viccaro et al., issued Jun. 27, 1995; U.S. Pat. No. 5,188,822, Viccaro et al., issued Feb. 23, 1993. None of the above references, however, teach that the incorporation of the silicone agents of the present invention into a non-aqueous denture adhesive composition also comprising an effective adhesive amount of a denture adhesive component, will provide antiplaque, antistain, and/or antideposition efficacy, especially in a non-aqueous matrix.

In accordance with the present invention, it has been discovered that denture adhesive compositions comprising an adhesive component with an antiplaque/antistain agent, provide excellent hold as well as antiplaque, antistain, and/or antideposition efficacy for the denture wearer.

### SUMMARY OF THE INVENTION

The present invention relates to a denture adhesive composition comprising:

a. a safe and effective adhesive amount of denture adhesive component;

b. a safe and effective amount of an antiplaque/antistain agent selected from the group consisting of dimethicone copolyol, silicone surfactant, aminoalkylsilicone, organosiloxane resin, and mixtures thereof; and

c. a non-aqueous denture adhesive carrier.

In addition the present invention relates to denture adhesive compositions comprising the above composition and at least one non-adhesive self-supporting layer. The present denture adhesive compositions can optionally comprise one or more additional adhesive components. The present invention further relates to a method of increasing the antiplaque, antistain, and/or antideposition efficacy or effect in the oral cavity of a denture wearer, in need thereof, by applying the above composition to the oral cavity.

### DETAILED DESCRIPTION OF THE INVENTION

A detailed description of essential and optional components of the present invention is given below.

Definitions

The term “safe and effective adhesive amount” as used herein means an amount sufficient to provide adherence to the oral cavity and/or adherence of a dental prosthesis to the palate and ridge of the oral cavity, without toxicity to the user, damage to oral tissue, and alteration of the denture material. The term “safe and effective amount” as used herein means an amount of an ingredient (e.g. antiplaque agent) high enough to significantly (positively) modify the condition to be treated, but low enough to avoid serious side effects (at a reasonable benefit/risk ratio), within the scope of sound medical judgment. The safe and effective amount of an ingredient (e.g. antiplaque agent) will vary with the particular condition being treated, the age and physical condition of the patient being treated, the severity of the condition, the duration of treatment, the nature of concurrent therapy, the specific form of the antiplaque agent employed, and the particular vehicle from which the antiplaque agent is applied.

The term “AVE/MA” as used herein refers to alkyl vinyl ether-maleic acid copolymer. The term “mixed polymer salts” or “mixed salts” as used herein refers to salts of the alkyl vinyl ether-maleic acid or anhydride copolymers where at least 2 different cations are mixed on the same polymer with each other or with other ester functions. The present invention, in another embodiment, comprises mixed polymer salts containing zinc cations.

The term “free acid” (“FA”) component as used herein refers either to the unreacted carboxyl groups COOH) of the alkyl vinyl ether-maleic acid polymer or to the unreacted carboxyl groups (—COOH) of the alkyl vinyl ether-maleic acid polymer plus any other monovalent cations of carboxyl groups; e.g. —COONa, of the polymer. Monovalent cations include Group IA cations, such as sodium, potassium, hydrogen, etc. In another embodiment, the term “free acid” refers to the unreacted carboxyl groups (—COOH) of the alkyl vinyl ether-maleic polymer, plus sodium and potassium cations. In yet another embodiment, the term “free acid” refers only to the unreacted carboxyl groups (—COOH) of the alkyl vinyl ether-maleic polymer.

The percentages used herein to describe the cationic salt function of the alkyl vinyl ether-maleic acid or anhydride copolymers are defined as the stoichiometric percent of the total initial carboxyl groups reacted on the polymer. All other percentages used herein are by weight of the composition unless otherwise indicated. All measurements referred to herein are made at 25° C. unless otherwise specified. All percentages, ratios, and levels of ingredients referred to herein are based on the actual amount of the ingredient, and do not include solvents, fillers, or other materials with which the ingredient may be combined as a commercially available product, unless otherwise indicated.

All publications, patent applications, and issued patents mentioned herein are hereby incorporated in their entirety by reference. Citation of any reference is not an admission regarding any determination as to its availability as prior art to the claimed invention.

Herein, “comprising” encompasses the terms “consisting of” and “consisting essentially of”.

### Denture Adhesive Component

The compositions of the present invention comprise a safe and effective adhesive amount of denture adhesive component. The adhesive component is present at a level of from about 0.1% to about 99.9%, in another embodiment from about 10% to about 70%, and in yet another embodiment from about 20% to about 50%, by weight of the composition.

The adhesive component includes natural gums, synthetic polymeric gums, alkyl vinyl ether maleic acid copolymer, alkyl vinyl ether anhydride copolymer, salts of alkyl vinyl ether maleic acid copolymer, synthetic polymers, mucoadhesive polymers, water-soluble hydrophilic colloids or polymers having the property of swelling upon exposure to moisture to form a mucilaginous mass, hydrophilic polymers, saccharide derivatives, cellulose derivatives, and mixtures thereof. In one embodiment the adhesive component is alkyl vinyl ether-maleic acid polymer and salts thereof, cellulose derivatives, karaya gum, guar gum, gelatin, algin, sodium alginate, tragacanth, chitosan, polyethylene glycol, acrylamide polymers, carbopol, polyvinyl alcohol, polyamines, polyquarternary compounds, polybutenes, silicones, ethylene oxide polymers, polyvinylpyrrolidone, cationic polyacrylamide polymers, and mixtures thereof. In another embodiment the adhesive component is alkyl vinyl ether-maleic acid polymer and salts thereof, cellulose derivatives, polyethylene glycol, polyethylene oxide, karaya gum, sodium alginate, chitosan, polyvinyl alcohol, and mixtures thereof. In yet another embodiment the adhesive component is alkyl vinyl ether-maleic acid polymer and salts thereof, cellulose derivatives such as methylcellulose, carboxymethylcellulose, hydroxyethylcellulose, hydroxypropylcellulose, hydroxy-propylmethylcellulose, and mixtures thereof.

Alkyl Vinyl Ether-Maleic Copolymer

The alkyl vinyl ether-maleic acid polymer consists essentially of the repeated structural unit:

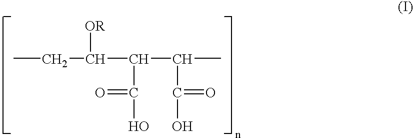

wherein R represents an alkyl radical, in another embodiment a C, to C₅ alkyl radical, n is an integer greater than one representing the number of repeated occurrences of the structural unit in a molecule of the polymer.

The alkyl vinyl ether-maleic acid or anhydride copolymers useful in the present invention are preferably in the mixed salt form, comprising a cationic salt function. In one embodiment the adhesive component is a salt of an alkyl vinyl ether-maleic acid or anhydride copolymer wherein the copolymer contains a cationic salt function comprising a cation selected from the group consisting of Group IA and Group 2A cations of the periodic table, yttrium, titanium, zirconium, vanadium, chromium, manganese, iron, nickel, copper, zinc, boron, aluminum, cations and mixtures thereof, in another embodiment strontium, zinc, iron, boron, aluminum, vanadium, chromium, manganese, nickel, copper, yttrium, titanium, magnesium, calcium, sodium, cations and mixtures thereof; in yet another embodiment, strontium, zinc, iron, magnesium, calcium, sodium, cations, and mixtures thereof.

In another embodiment the mixed salt contains a cationic salt function comprising from about 5% to about 50%, in another embodiment from about 10% to about 40%, in yet another embodiment from about 10% to about 35% (of the total initial carboxyl groups reacted) zinc cations. These zinc cations can be mixed with other cations selected from the group consisting of: from about 5% to about 65%, in another embodiment from about 10% to about 60%, strontium cations; from about 0.001% to about 2.5%, in another embodiment from about 0.01% to about 2% of iron, boron, aluminum, vanadium, chromium, manganese, nickel, copper, yttrium, and/or titanium; from about 5% to about 65%, in another embodiment from about 15% to about 50% of calcium, zirconium, and/or magnesium.

Other AVE/MA salts or acids can be used in the present invention and are disclosed in: U.S. Pat. No. 6,239,191, issued May 29, 2001, Wong et al.; U.S. Pat. No. 5,073,604, issued Dec. 17, 1991, Holeva et al.; U.S. Pat. No. 5,424,058, issued Jun. 13, 1995, Rajaiah et al.; U.S. Pat. No. 5,525,652, issued Jun. 11, 1996, Clarke et al.; U.S. Pat. No. 4,758,630, issued Jul. 19, 1988, Shah et al.; U.S. Pat. No. 5,304,616, issued Apr. 19, 1994, Rajaiah et al.; U.S. Pat. No. 5,830,933, issued Nov. 3, 1998, Synodis et al.; U.S. Pat. No. 3,003,988, issued Oct. 10, 1961, Germann et al.; U.S. Pat. No. 5,750,591, issued May 12, 1998, Clarke et al.; U.S. Pat. No. 5,872,161, issued Feb. 16, 1999, Liang et al., U.S. Pat. No. 5,750,591, Clarke et al., issued May 12, 1992; U.S. Pat. No. 5,525,652, Clarke et al., issued Jun. 11, 1996; U.S. Pat. No. 5,830,933, Synodis et al., issued Nov. 3, 1998; U.S. Pat. No. 6,025,411, Wong et al., issued Feb. 15, 2000; U.S. Pat. No. 2,047,398, issued Jul. 14, 1936, Voss et al.; U.S. Pat. No. 3,003,988, issued Oct. 10, 1961, Germann et al.; all of which are incorporated herein by reference in their entirety

In one embodiment the free acid level of the salt copolymer is at least about 36%, in yet another embodiment from about 36% to about 60%, and in yet another embodiment from about 40% to about 55%, of the total initial carboxyl groups of the copolymer.

The specific viscosity of the starting copolymer acid or copolymer anhydride is above 1.0, in another embodiment above 2.5, when preferably measured in a 1% weight/volume solution in MEK (methyl ethyl ketone) at 25° C. Other methods and solvents can be used to measure the specific viscosity such as a 1% weight/volume solution in DMF (dimethyl formamide) at 25° C. and a 1% weight/volume solution in 2-butanone at 25° C.

The alkyl vinyl ether maleic anhydride polymers may be readily obtained by methods described in U.S. Pat. No. 6,355,706B1, issued Mar. 12, 2002, Rajaiah et al., which is herein incorporated by reference.

The compositions of the present invention, in one embodiment, comprise a safe and effective adhesive amounts of the salt of AVE/MA or anhydride copolymers, in another embodiment at least 20 percent by weight, in another embodiment at least about 25% by weight, and in yet another embodiment at least 30 percent by weight of the composition, as the sole adhesive component or as a co-adhesive in joint usage with other adhesive components.

AntiplaQue/Antistain Agent

The antiplaque, antistain, antideposition agent is selected from the group consisting of dimethicone copolyol, silicone surfactant, aminoalkylsilicone, organosiloxane resin, and mixtures thereof. In another embodiment the antiplaque/antistain agent is selected from the group consisting of dimethicone copolyol, silicone surfactant, aminoalkylsilicone, and mixtures thereof; in another embodiment is selected from the group consisting of dimethicone copolyol, silicone surfactant, organosiloxane resin, and mixtures thereof; in yet another embodiment from the group consisting of silicone surfactant, aminoalkylsilicone, organosiloxane resin, and mixtures thereof; and in yet another embodiment from the group consisting of dimethicone copolyol, aminoalkylsilicone, organosiloxane resin, and mixtures thereof; and in yet another embodiment is selected from the group consisting of dimethicone copolyol.

Dimethicone Copolyol

The dimethicone copolyol is selected from alkyl- and alkoxy-dimethicone copolyols having the formula (II):

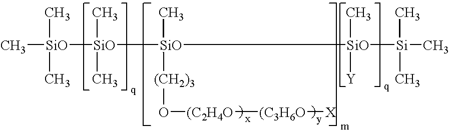

wherein X is selected from the group consisting of hydrogen, alkyl, alkoxy and acyl groups having from about 1 to about 16 carbon atoms, Y is selected from the group consisting of alkyl and alkoxy groups having from about 8 to about 22 carbon atoms, n is from about 0 to about 200, m is from about 1 to about 40, q is from about 1 to about 100, the molecular weight of the residue (C₂H₄O—)ₓ(C₃H₆O—)_{y}X is from about 50 to about 2000, in another embodiment from about 250 to about 1000 and x and y are such that the weight ratio of oxyethylene:oxypropylene is from about 100:0 to about 0:100, in another embodiment from about 100:0 to about 20:80. In preferred embodiments, the dimethicone copolyol is selected from C₁₂ to C₂₀ alkyl dimethicone copolyols and mixtures thereof. Highly preferred is cetyl dimethicone copolyol marketed under the Trade Name Abil®EM90, from Goldschmidt.

The dimethicone copolyol is generally present in a level of from about 0.001% to about 30%, in another embodiment from about 0.01% to about 5%, in yet another embodiment from about 0.1% to about 1.5% by weight.

Silicone Surfactant

The silicone surfactant of the present invention has the general formula (II) wherein X is selected from hydrogen, alkyl, alkoxy and acyl groups having from about 1 to about 16 carbon atoms, Y is CH₃, q is 0, n is from about 1 to about 100, m is from about 1 to about 40, the molecular weight of the residue (C₂H₄ O—)ₓ—(C₃H₆O—)_{y} X is from about 50 to about 2000, in another embodiment from about 250 to about 1,000, and x and y are such that the weight ratio of oxyethylene:oxypropylene is from about 100:0 to about 0:100, in another embodiment from about 100:0 to about 20:80.

In one embodiment, the silicone surfactant is selected from dimethicone copolyols having a HLB value of greater than 14 and mixtures thereof. In another embodiment the silicone surfactant is end-capped (X is alkyl, more particularly methyl) dimethicone copolyols, especially where the pendant side chain is all oxyethylene (y is 0), such as that marketed under the Trade Name Silwet L7600 (CAS Registry No. 689 38-54-5 from Union Carbide). In another embodiment the silicone surfactant is Silwet L7230, CAS Registry No. 689 37-55-3 from Union Carbide. The silicone surfactant is generally present in a level of from about 0.01% to about 25%, preferably from about 0.3% to about 10%, more preferably from about 0.5% to about 2% by weight. In one embodiment the silicone surfactant is used in combination with dimthicone copolyol, wherein, in general the ratio of silicone surfactant to dimethicone copolyol is from about 0.5:1 to 15:1, in another embodiment from about 1:1 to 10:1, and in yet another embodiment from about 2:1 to 8:1 by weight.

Aminoalkylsilicone

The aminoalkylsilicone is selected from noncyclic, hydrophobic aminoalkysilicones having a formula comprising two basic units:

1) (R¹)ₘ(R)ₙSiO_{(4-m-n)/2} wherein m+n is 1, 2 or 3; n is 1, 2 or 3; m is 0, 1, 2; and

2) (R¹)ₐ(R²)_{b}SiO_{(4-a-b)/2} wherein a+b is 1, 2, or 3, and a and b are integers,

wherein R¹ and R² are independently selected from the group consisting of hydrogen, alkyl and alkenyl of about 1 to about 10 carbons optionally substituted with fluoro or cyano groups, hydroxy, alkoxy, and acetoxy, for example, wherein R¹ and R² are independently selected from the groups consisting of methyl, ethyl, phenyl, vinyl, trifluoropropyl and cyanopropyl, and R is

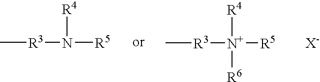

wherein R³ is a divalent alkylene of about 1 to about 20, in another embodiment about 3 to about carbon atoms optionally substituted or interrupted by 0 atoms, R⁴, R⁵ and R⁶ which may be the same or different are selected from the groups consisting of hydrogen, alkyl of about 1 to about 20, in another embodiment about 1 to about 10, in yet another embodiment about 1 to about 4 carbons optionally substituted or interrupted by N atoms, 0 atoms, or mixtures of N and 0 atoms, and X⁻ is a monovalent anion such as halide, hydroxide, and tosylate, said aminoalkylsilicone including about 60% or less, in another embodiment from about 0.1-30%, in yet another embodiment from about 0.2-10% and especially from about 0.5-2% of unit (1) on a repeating unit basis.

In one embodiment, the aminoalkylsilicones comprise amodimethicones. Amodimethicones are polydimethylsiloxane polymers containing aminoalkyl groups. The aminoalkyl groups may be present either pendant or at one or more ends of the polydimethylsiloxane chain. Preferred are aminoalkylsilicones in which aminoalkyl moiety R is selected from (CH₂)₃NH₂, (CH₂)₃NHCH₂CH₂NH₂, (CH₂)₃N(CH₂CH₂OH)₂, (CH₂)₃NH₃⁺X⁻ and (CH₂)₃N(CH₃)₂(C₁₈H₃₇)⁺X⁻, and especially from (CH₂)₃NH₂ and (CH₂)₃NHCH₂CH₂NH₂. In one embodiment the aminoalkyl silicones have an average molecular weight of about 5,000 and above, in another embodiment from about, 5000 to about 100,000, in yet another embodiment from about 5,000 to about 30,000. Aminoalkylsilicone compounds suitable for use herein are well known. Methods of preparing aminoalkylsilicones are given in, for example, U.S. Pat. No. 2,930,809, which is incorporated herein by reference in its entirety. Examples of amodimethicones include Dow Corning's DC-929, DC-Q2-7224 and Q2-8075 and OSI's Magnasoft fluid, the latter being preferred. These polymers comprise aminoalkyl groups affixed to a predominantly polydimethylsiloxane structure. The typical structure of Magnasoft's aminoalkyl group-containing units is

—OSi(Me)C₃H₆NHCH2CH₂NH₂

The aminoalkylsilicone is is generally present in a level of from about 0.001% to about 30%, in another embodiment from about 0.01% to about 10%, in yet another embodiment from about 0.1% to about 2% by weight.

Organosiloxane Resins

Silicone resins are highly crosslinked polymeric siloxane systems. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. In one embodiment, the ratio of oxygen:silicon atoms is at least about 1.2:1.0.

Silicone materials and silicone resins in particular can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO)_{1.5}; D denotes the difunctional unit (CH₃)₂SiO; T denotes the trifunctional unit (CH₃)SiO_{1.5}; and Q denotes the quadra- or tetra-functional unit SiO₂. Note that a small amount, up to about 5% of silanol or alkoxy functionality may also be present in the resin structure as a result of processing.

Primes of the unit symbols, e.g., M′, D′, T′, and Q′, denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or M′ in a silicone resin is indicative of higher levels of crosslinking. As discussed before, however, the overall level of crosslinking can also be indicated by the oxygen to silicon ratio.

The organosiloxane resins are solid at about 25° C. and the average molecular weight of the resins is from about 1,000 to about 10,000. The resins are soluble in organic solvents such as toluene, xylene, isoparaffins, cyclosiloxanes and mixtures thereof.

In one embodiment the silicone resins used herein are MQ, MT, MTQ, and MDTQ resins. MQ resins are disclosed in U.S. Pat. No. 5,330,747, Krzysik, issued Jul. 19, 1994. Thus, in one embodiment the silicone substituent is methyl. In another embodiment the resin are MQ resins wherein the M:Q ratio is from about 0.5:1.0 to about 1.5:1.0. Organosiloxane resins such as these are commercially available, for example, Wacker 803 and 804 available from Wacker Silicones Corporation of Adrian, Mich., US, and G.E. 1170-002 (SR 1000) from the General Electric Company.

Generally, the level of resin used in the present invention is from about 0.1% to about 50%, preferably from about 0.5% to about 30%, and even more preferably from about 1% to about 10%.

Fluid Diorganopolysiloxane-Based Polymers

In addition to the organosiloxane resins disclosed above, the compositions of the present invention may optionally further comprise a fluid diorganopolysiloxane-based polymer to be combined with the organosiloxane resins. Said fluid diorganopolysiloxane-based polymers useful in the present invention span a large range of viscosities; from about 10 to about 10,000,000 centistokes (cSt) at 25° C. Some diorganopolysiloxane polymers useful in this invention exhibit viscosities greater than 10,000,000 centistokes (cSt) at 25° C. and therefore are characterized by manufacturer specific penetration testing. Examples of this characterization are GE silicone materials SE 30 and SE 63 with penetration specifications of 500-1500 and 250-600 (tenths of a millimeter), respectively.

Among the fluid diorganopolysiloxane-based polymers of the present invention are diorganopolysiloxane polymers comprising repeating units, where said units correspond to the formula (R₂SiO)ₙ, where R is a monovalent radical containing from 1 to 6 carbon atoms, in one embodiment they are selected from the group consisting of methyl, ethyl, propyl, isopropyl, butyl, isobutyl, t-butyl, amyl, hexyl, vinyl, allyl, cyclohexyl, amino alkyl, phenyl, fluoroalkyl and mixtures thereof. The fluid diorganopoylsiloxane polymers employed in the present invention may contain one or more of these radicals as substituents on the siloxane polymer backbone. The fluid diorganopolysiloxane polymers may be terminated by triorganosilyl groups of the formula (R′₃Si) where R′ is a monovalent radical selected from the group consisting of radicals containing from 1-6 carbon atoms, hydroxyl groups, alkoxyl groups and mixtures thereof.

Silicone gum corresponds to the formula:

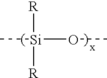

where R is a methyl group.

Fluid diorganopolysiloxane polymers such as these are commercially available, for example, SE 30 silicone gum and SF96 silicone fluid available from the General Electric Company. Similar materials can also be obtained from Dow Corning and from Wacker Silicones.

In one embodiment of the present invention, the ratio of organosiloxane resin to fluid diorganopolysiloxane-based polymer is from about 10:1 to about 1:10, in another embodiment from about 2:1 to about 8:1, and in yet another embodiment is from about 4:1 to about 6:1.

Non-Aqueous Vehicle

The level of non-aqueous vehicle is from 10% to about 90%, in another embodiment is from about 20% to about 80%, and in yet another embodiment is from about 20% to about 60%, by weight of the composition.

The non-aqueous vehicle is generally any chemical in any physical form that contains less than about 3%, in another embodiment contains less that about 1% water, and in another embodiment is essentially free of water. In one embodiment the non-aqueous vehicle is selected from the group consisting of liquid petrolatum, petrolatum, mineral oil, silicone oil, polybutene, glycerin, natural and synthetic oils, fats, polyvinylacetate, natural and synthetic waxes such as animal waxes like beeswax, lanolin and shellac, hydrocarbons, hydrocarbon derivatives, vegetable oil waxes such as carnauba, candelilla and bayberry wax, vegetable oils such as caprylic/capric triglycerides, in another embodiment is selected from the group consisting of liquid petrolatum, petrolatum, mineral oil, vegetable oils such as corn, soy bean, cottonseed, castor, palm and coconut oils and animal oil such as fish oil and oleic acid, and mixtures thereof; and in yet another embodiment is mineral oil or petrolatum.

Optional Non-Adhesive Self-Supporting Layer

The present denture adhesive compositions optionally comprise at least one non-adhesive self-supporting layer. The non-adhesive self-supporting layer is characterized by its ability to maintain strength and provide integrity for the adhesive composition in the presence of water and/or saliva. The non-adhesive self-supporting layer may include materials such as polyester, polypropylene, nylon, rayon, cellulose acetate, non-adhesive cellulose derivatives, cloth, fibrous fleece, paper, plastic, leather, microcrystalline wax, synthetic fibers, natural fibers, and mixtures thereof. Preferred are non-adhesive cellulose derivatives, polyester, polypropylene, nylon, rayon, cloth, paper, microcrystalline wax, and mixtures thereof. More preferred are polyester, polypropylene, rayon, nylon, cloth and paper, and mixtures thereof.

The non-adhesive self-supporting layer may be in any physical form suitable for providing strength and/or integrity to the present adhesive compositions. Such physical forms include non-woven, woven, continuous, chopped, and combinations thereof. In addition, the non-adhesive self-supporting layer may be formed by any process commonly known in the art. Such processes include un-bonded, spraybonded, spun-bonded, needle-punched, carded, thermal bonded hydroentangled, meltblown, aperture print bonded, needled, wet-laid, dry-laid, and combinations thereof.

Other Optional Ingredients

In addition one or more toxicologically-acceptable plasticizers may also be included in the present compositions. The term “toxicologically-acceptable”, as used herein, is used to describe materials that are suitable in their toxicity profile for administration to humans and/or lower animals. Plasticizers that may be used in the present compositions include dimethyl phthalate, diethyl phthalate, dioctyl phthalate, glycerin, diethylene glycol, triethylene glycol, Igepal®, Gafac®, sorbitol, tricresyl phosphate, dimethyl sebacate, ethyl glycolate, ethylphthalyl ethyl glycolate, o- and p-toluene ethyl sulfonamide, and mixtures thereof. Plasticizers may be present at a level of from about 0% to about 70%, in another embodiment from about 1% to about 30%, by weight of the compositions.

Other suitable ingredients include colorants, preservatives such as methyl and propyl parabens; thickeners such as silicon dioxide, and polyethylene glycol. Preferred are polyethylene glycol and silicon dioxide. Colorants, preservatives, and thickeners may be present at levels of from about 0% to about 20%, in another embodiment from about 1% to about 10%, by weight of the composition.

Flavors, Fragrance, Sensates

The compositions of the present invention may also include one or more components which provide flavor, fragrance, and/or sensate benefit (warming or cooling agents). Suitable components include natural or artificial sweetening agents, menthol, menthyl lactate, wintergreen oil, peppermint oil, spearmint oil, leaf alcohol, clove bud oil, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof, as well as coolants. The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosure of both are herein incorporated by reference in their entirety. These agents may be present at a level of from about 0% to about 50%, by weight of the composition.

Process for Preparation of the Composition

A process for preparing denture adhesive compositions of the present invention (creams, powders, wafers, liquids, aerosols, pastes) comprises conventional methods disclosed in the art. Conventional methods are taught in U.S. Pat. No. 5,525,652, issued Jun. 11, 1996, Clarke et al.; U.S. Pat. No. 3,003,988, issued Oct. 10, 1961, Germann et al.; U.S. Pat. No. 5,073,604, Holeva et al., issued Dec. 17, 1991; and U.S. Pat. No. 5,872,161, Liang et al., issued Feb. 16, 1999, all of which are herein incorporated by reference in their entirety.

A process for the preparation of the present denture adhesive compositions optionally comprising a non-adhesive self-supporting layer, comprises coating a weighed amount of the adhesive components onto the non-adhesive self-supporting layer. This process is disclosed in U.S. Pat. No. 5,877,233, Liang et al, issued Mar. 2, 1999; U.S. Pat. No. 5,872,160, issued Feb. 16, 1999, Liang et al.; U.S. Pat. No. 5,880,172, Rajaiah et al., filed Oct. 25, 1996, all of which are incorporated herein by reference in their entirety.

### Method of Using the Composition

The present invention further relates to a method of reducing plaque and stain in the oral cavity of a denture wearer, in need thereof, by administering the above denture adhesive compositions to the oral cavity. The adhesive compositions may be in the form of a powder, cream, paste, liquid, aerosol, and/or wafer. Powder forms are sprinkled on a dental prosthesis, moistened and then inserted into the oral cavity. The compositions may also be combined with various conventional delivery vehicles to form liquids or pastes which are applied to a dental prosthesis and inserted into the oral cavity. These compositions can optionally comprise at least one non-adhesive self-supporting layer. Denture adhesive compositions with a self supporting layer are thoroughly moistened and applied to dentures. The above compositions are applied to dentures, directly to the oral cavity, palate or ridge of the oral cavity, or applied to both, and thereafter the dentures are secured to the ridge or palate of the oral cavity.

The following examples further describe and demonstrate embodiments within the scope of the present invention. The examples are given solely for the purpose of illustration and are not to be construed as limitations of the present invention. Many variations of these are possible without departing from the spirit and scope of the invention.

### EXAMPLE I

Denture stabilizing compositions in cream form can be made by blending together the following ingredients:

| | A | B | C | D |
|---|---|---|---|---|
| | weight | weight | weight | weight |
| | (grams) | (grams) | (grams) | (grams) |
| White Mineral Oil | 23.93 | 23.93 | 23.93 | 0 |
| Petrolatum, White | 21.77 | 20.87 | 11.87 | 0 |
| Carboxymethylcellulose Sodium | 20.00 | 20.00 | 20.00 | 20.00 |
| Silicon Dioxide, Colloidal | 1.14 | 1.14 | 1.14 | 1.14 |
| Colorant | 0.06 | 0.06 | 0.06 | 0.06 |
| Antiplaque/antistain agent¹ | 0.1 | 1.00 | 10.00 | 45.8 |
| Any MVE/MA Copolymer Salt | 33.00 | 33.00 | 33.00 | 33.00 |

| | E | F |
|---|---|---|
| | weight | weight |
| | (grams) | (grams) |
| White Mineral Oil | 15.9 | 4 |
| Petrolatum, White | 27.85 | 27.85 |
| Carboxymethylcellulose Sodium | 24 | 24 |
| Silicon Dioxide, Colloidal | 1.10 | 1.10 |
| Colorant | 0.05 | 0.05 |
| Antiplaque/antistain agent² | 0.1 | 10.0 |
| Any MVE/MA Copolymer Salt | 30 | 30 |
| Alkyl vinylether maleic acid copolymer | 1 | 3 |

¹Any one of Abil EM90, Magnasoft Fluid, Wacker 803, Silwet L7600, or mixtures thereof.
²Any one of Abil EM90, Magnasoft Fluid, Wacker 803, Silwet L7600, or mixtures thereof.

Weigh, heat and mix the red dye, antiplaque/antistain agent, petrolatum, and mineral oil in a glass jar at 50 to 60° C. until visually uniform. Then weigh and shake-blend the powders (colloidal silicon dioxide, CMC, AVE/MA copolymer salt, and AVE/MA acid copolymer) together in a container. Thereafter, mix the powders into the liquid with a spatula until visually a uniform pink cream. The above cream can be modified by increasing or decreasing the level of AVE/MA salt by 0 to 10 grams, petrolatum by 0 to 10 grams, and/or the CMC by 0 to 10 grams. The above cream composition can also be modified by using mixtures of the various AVE/MA mixed polymer salts and/or acid. The composition can also be further modified substituting another antiplaque/antistain agent such as DC-929, DC Q2 7224, Q2 8075, Wacker 804, GE 1170-002(SR 1000), and mixture thereof. The subject places from 0.1 to 2 grams of the cream composition on the denture. Then the subject inserts the denture into his/her mouth and presses it into place.

### EXAMPLE II

Denture stabilizing compositions in powder form can be made by blending together the following ingredients:

| | A | B | C |
|---|---|---|---|
| | weight | weight | weight |
| | (grams) | (grams) | (grams) |
| Carboxymethylcellulose Sodium | 40.00 | 40.00 | 40.00 |
| Antiplaque/antistain agent³ | 0.10 | 1.00 | 10.00 |
| Any MVE/MA Copolymer Salt | 59.90 | 59.00 | 50.00 |

³Any one of Abil EM90, Magnasoft Fluid, Wacker 803, Silwet L7600, or mixtures thereof.

Blend all components together with spraying of the antiplaque agent if necessary. The above compositions can be modified by increasing or decreasing the AVE/MA mixed salt by 0 to 50 grams and/or the CMC by 0 to 40 grams. The above powder compositions can also be modified by using mixtures of the various AVE/MA mixed salts and/or acid. The subject places from 0.1 to 2 grams of the composition on a pre-moistened denture, allowing it to hydrate briefly. Then the subject inserts the denture into his/her mouth and presses it into place.

### EXAMPLE III

Denture stabilizing compositions in wafer form can be made by wetting a 58″ by 13.3″ non-woven polyester (non-adhesive self-supporting layer) with water. Uniformly coat this wet sheet with the compositions listed above in Example II. Thereafter, rewet the layer with water. Dry the layer. Mechanically soften the composition by ring-roller, and then smooth the composition on a hydraulic press. Die-cut the composition into desired shapes. Moisten and apply these wafer compositions to the dentures. Then insert the denture into the mouth and press it into place.

These wafer compositions can be modified by increasing or decreasing the AVE/MA mixed polymer salt by 0 to 60 grams and/or CMC by 0 to 60 grams. The above wafer compositions can also be modified by using mixtures of the various AVE/MA mixed salts and/or acids.

What is claimed is:

1. A denture adhesive composition comprising:
a. a safe and effective adhesive amount of denture adhesive component;
b. a safe and effective amount of an antiplaque or antistain agent selected from the group consisting of dimethicone copolyol, silicone surfactant, aminoalkylsilicone, organosiloxane resin, and mixtures thereof; and
c. a non-aqueous denture adhesive carrier.

2. The composition of claim 1 wherein the antiplaque or antistain agent is alkyl- and alkoxy-dimethicone copolyols having the formula (II): 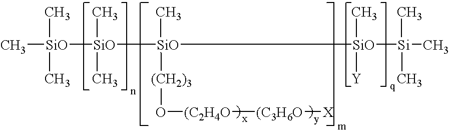
wherein X is selected from the group consisting of hydrogen, alkyl, alkoxy and acyl groups having from about 1 to about 16 carbon atoms, Y is selected from the group consisting of alkyl and alkoxy groups having from about 8 to about 22 carbon atoms, n is from 0 to about 200, m is from about 1 to about 40, q is from about 1 to about 100, the molecular weight of the residue (C₂H₄O—)ₓ(C₃H₆O—)_{y}X is from about 50 to about 2000, and x and y are such that the weight ratio of oxyethylene:oxypropylene is from about 100:0 to about 0:100.

3. The composition of claim 2 wherein the antiplaque agent is present at a level of from about 0.01% to about 5% and is selected from C₁₂ to C₂₀ alkyl dimethicone copolyols and mixtures thereof.

4. The composition of claim 3 wherein the antiplaque agent is cetyl dimethicone copolyol.

5. The composition of claim 1 wherein the antiplaque or antistain agent is a silicone surfactant having formula (II): 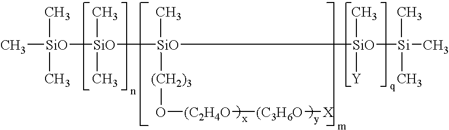
wherein X is selected from hydrogen, alkyl, alkoxy and acyl groups having from about 1 to about 16 carbon atoms, Y is CH₃, q is 0, n is from about 1 to about 100, m is from about 1 to about 40, the molecular weight of the residue (C₂H₄ O—)ₓ(C₃H₆ O—)_{y}X is from about 50 to about 2000, and x and y are such that the weight ratio of oxyethylene:oxypropylene is from about 100:0 to about 0:100.

6. The composition of claim 5 wherein the silicone surfactant is present at a level of from about 0.01% to about 10% by weight and is selected from the group consisting of dimethicone copolyols of the general formula II wherein the end capping group X is methyl and y is 0, and mixtures thereof.

7. The composition of claim 1 wherein the antiplaque or antistain agent is a noncyclic, hydrophobic aminoalkysilicones having a formula comprising two basic units:
1) (R¹)ₘ(R)ₙSiO_{(4-m-n)/2} wherein m+n is 1, 2 or 3; n is 1, 2 or 3; m is 0, 1, 2; and
2) (R¹)ₐ(R²)_{b}SiO_{(4-a-b)/2} wherein a+b is 1, 2, or 3, and a and b are integers,
wherein R¹ and R² are independently selected from the group consisting of hydrogen, alkyl and alkenyl of about 1 to about 10 carbons, hydroxy, alkoxy, and acetoxy, and R is 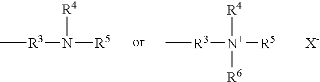
wherein R³ is a divalent alkylene of about 1 to about 20, carbon atoms optionally substituted or interrupted by O atoms, R⁴, R⁵ and R⁶ which may be the same or different are selected from the group consisting of hydrogen, alkyl of about 1 to about 20, carbons optionally substituted or interrupted by N, O, or mixtures of N and O atoms and X⁻ is a monovalent anion said aminoalkylsilicone including about 60% or less of (1) on a repeating unit basis.

8. The composition of claim 7 wherein the antiplaque agent is amodimethicones and is present at a leve of from about 0.01% to about 10% by weight.

9. The composition of claim 8 wherein the antiplaque agent is an aminoalkylsilicone
wherein R is (CH₂)₃ NH₂ or (CH₂)₃ NHCH₂CH₂NH₂.

10. The composition of claim 1 wherein the antiplaque or antistain agent is a organosiloxane resins selected from the group consisting of MQ, MT, MTQ, and MDTQ, and mixtures thereof.

11. The composition of claim 10 wherein the antiplaque agent is MQ resin having an M:Q ratio from about 0.5:1.0 to about 1.5:1.0.

12. The composition of claim 1 wherein the denture adhesive component is selected from the group consisting of natural gums, synthetic polymeric gums, alkyl vinyl ether maleic acid copolymer, alkyl vinyl ether anhydride copolymer, salts of alkyl vinyl ether maleic acid or anhydride copolymer, cellulose derivatives, and mixtures thereof.

13. The composition of claim 12 wherein the denture adhesive component is at a level of from about 15% to about 60% by weight.

14. The composition of claim 13 wherein the denture adhesive component is selected from the group consisting of an alkyl vinyl ether-maleic acid copolymer, salts of an alkyl vinyl ether-maleic acid or anhydride copolymer, cellulose derivatives, and mixtures thereof.

15. The composition of claim 14 wherein the adhesive component is a salt of an alkyl vinyl ether-maleic acid or anhydride copolymer, the salt containing a cationic salt function comprising a cation selected from the group consisting of Group IA and Group 2A cations of the periodic table, yttrium, titanium, zirconium, vanadium, chromium, manganese, iron, nickel, copper, zinc, boron, aluminum, sodium, and mixtures thereof.

16. The denture adhesive composition according to claim 15 wherein the cations are selected from the group consisting of strontium, zinc, iron, magnesium, calcium, sodium, zirconium, and mixtures thereof.

17. The denture adhesive composition according to claim 1 further comprising one or more ingredients selected from the group consisting of plasticizers, colorants, preservatives, thickeners, flavors, fragrances, sensates, and mixtures thereof.

18. The denture adhesive composition according to claim 16 wherein the non-aqueous carrier is selected from the group consisting of petrolatum, liquid petrolatum, mineral oil, glycerin, polybutene, and mixtures thereof.

19. The denture adhesive composition according to claim 1 wherein the composition further comprises at least one non-adhesive self-supporting layer.

20. The denture adhesive composition according to claim 19 wherein the non-adhesive self-supporting layer is selected from the group consisting of polyester, polypropylene, nylon, rayon, cellulose acetate, non-adhesive cellulose derivatives, cloth, fibrous fleece, paper, plastic, leather, microcrystalline wax, synthetic fibers, natural fibers, and mixtures thereof.

21. A method of providing antiplaque, antistain, or antideposition effectiveness in the oral cavity of a denture wearer in need thereof, by applying the denture adhesive composition of claim 1 to the oral cavity.
